(19) United States
(12) Patent Application Publication
(10) Pub. No.: US 2001/0041185 A1
(43) Pub. Date: Nov. 15, 2001

(54) Pre-powdered cosmetic pad

(75) Inventor: Frank LoManto, Farmingdale, NY (US)

Correspondence Address: Charles R. Hoffmann, Esq., HOFFMANN & BARON, LLP, 6900 Jericho Turnpike, Syosset, NY, 11791, US

(73) Assignee: Penthouse Manufacturing Co., Inc.

(21) Appl. No.: 09/847,750

(22) Filed: May 2, 2001

Related U.S. Application Data
(60) Provisional application No. 60/204,350, filed on May 15, 2000.

(51) Int. Cl. A61K 7/00; B32B 5/02

(52) U.S. Cl. 424/401; 442/123; 427/1

(57) ABSTRACT

A method and apparatus for embedding an amount of cosmetic powder into a cosmetic pad, and a pre-powdered cosmetic pad thereof. The cosmetic pad generally includes a thin open-celled foam pad having a top face and a bottom face and an amount of cosmetic powder embedded into the top face of the pad so that the cosmetic powder is visible on the top face of the pad. In a method for embedding an amount of cosmetic powder into a cosmetic pad, a cosmetic pad is inserted into a chamber having an amount of cosmetic powder deposited therein and a pressure is applied within the chamber whereby an amount of cosmetic powder is drawn into the cosmetic pad and embedded therein. Preferably, a stencil having a decorative cut-out pattern is also inserted into the chamber between the cosmetic pad and the amount of cosmetic powder whereby, upon the application of pressure within the chamber, an amount of cosmetic powder is drawn through the stencil and embedded into the cosmetic pad. The apparatus generally includes a chamber having an inlet, an outlet and a cosmetic pad receiving space disposed between the inlet and the outlet. An amount of cosmetic powder is deposited within the chamber between the inlet and the cosmetic pad receiving space and a pressure source is connected to one or both of the inlet and the outlet for applying a pressure within the chamber to draw an amount of the cosmetic powder into a cosmetic pad inserted in the cosmetic pad receiving space. Preferably, a stencil having a decorative cut-out pattern is disposed between the cosmetic pad receiving space and the amount of cosmetic powder so that the cosmetic powder is drawn through the stencil into the pad in a decorative design.

### CROSS-REFERENCE TO RELATED APPLICATIONS

This application claims the benefit of U.S. Provisional Application No. 60/204,350, filed May 15, 2000.

### FIELD OF THE INVENTION

The present invention relates generally to a cosmetic applicator and more particularly to a cosmetic pad having an amount of powder embedded in the surface of the pad and a method for embedding the powder into the pad.

### BACKGROUND OF THE INVENTION

Cosmetic powder puffs for applying powders such as makeup or talc powder to the human skin are well known in the field. Such powder puffs are conventionally carried by the user for occasional use and comprise a body portion made of a material through which can pass small quantities of powder for spreading on the skin of the user.

Cosmetic pads having a powder or a liquid cosmetic product pre-applied or pre-embedded therein are also generally known. For example, GB 474,732 describes a powder puff comprising two layers between which a powder is encapsulated. The two foam layers are joined together by a radial seam around the periphery of the pad after the powder is sandwiched therebetween. U.S. Pat. No. 2,204,202 describes a pad comprising a circular sheet of foam having a notch cut in a peripheral surface of the pad into which a cosmetic product can be inserted. U.S. Pat. No. 5,762,946 discloses a foam pad comprising two foam layers between which a topical treatment product is encapsulated. The product may be placed between the layers prior to sealing the peripheral edges of the layers or, alternatively, the pad may be impregnated with a solution or an emulsion of the desired product and then dehydrated.

The above-disclosed pads all require a rather complicated manufacturing process for encapsulating the cosmetic product within the pad. Accordingly, it would be desirable to provide a simple and cost efficient manufacturing process for embedding or encapsulating a cosmetic powder within a pad prior to use.

### SUMMARY OF THE INVENTION

The present invention is a pre-powdered cosmetic pad having an amount of a powder embedded within the pad. The cosmetic pad generally includes a thin open-celled foam pad having a top face and a bottom face and an amount of cosmetic powder embedded into the top face of the pad so that the cosmetic powder is visible on the top face of the pad. Preferably, the cosmetic powder is embedded into the top face of the pad in a decorative pattern and an impermeable envelope is provided for containing the pad prior to use.

In a method for embedding an amount of cosmetic powder into a cosmetic pad, a cosmetic pad is inserted into a chamber having an amount of cosmetic powder deposited therein and a pressure is applied within the chamber whereby an amount of cosmetic powder is drawn into the cosmetic pad and embedded therein. The pressure applied within the chamber may be a negative pressure or a positive pressure. Preferably, a stencil is also inserted into the chamber between the cosmetic pad and the amount of cosmetic powder whereby, upon the application of pressure within the chamber, an amount of cosmetic powder is drawn through the stencil and embedded into the cosmetic pad. The stencil may include a decorative cutout pattern for embedding the cosmetic powder into the cosmetic pad in a decorative design.

The present invention further involves an apparatus for embedding an amount of powder into a cosmetic pad. The apparatus generally includes a chamber having an inlet, an outlet and a cosmetic pad receiving space disposed between the inlet and the outlet. The cosmetic pad receiving space is sized to receive at least a cosmetic pad inserted between the inlet and the outlet. An amount of cosmetic powder is deposited within the chamber between the inlet and the cosmetic pad receiving space and a pressure source is connected to one or both of the inlet and the outlet for applying a pressure within the chamber to draw an amount of the cosmetic powder into a cosmetic pad inserted in the cosmetic pad receiving space. Preferably, a stencil is disposed between the cosmetic pad receiving space and the amount of cosmetic powder so that the cosmetic powder is drawn through the stencil into the pad. Thus, if the stencil includes a decorative cutout pattern, the cosmetic powder can be embedded into the cosmetic pad in a decorative design. The pressure source can apply a negative pressure to the outlet of the chamber or can apply a positive pressure to the inlet of the chamber. The chamber may also include a screen disposed over at least one of the inlet and the outlet for preventing the cosmetic powder from escaping the chamber.

A preferred embodiment of a pre-powdered cosmetic pad and a method and apparatus for manufacturing same, as well as other features and advantages of this invention will be apparent from the following detailed description, which is to be read in conjunction with the accompanying drawings.

### BRIEF DESCRIPTION OF THE DRAWINGS

FIG. 1 is a cross sectional view of the vacuum chamber for manufacturing the pre-powdered cosmetic pad;

FIG. 2 is a top perspective view of a pre-powdered cosmetic pad formed in accordance with the present invention; and

FIG. 3 is a top perspective view of a pre-powdered cosmetic pad formed in accordance with the present invention showing an ornamental design embedded on the surface of the pad.

### DETAILED DESCRIPTION OF THE PREFERRED EMBODIMENT

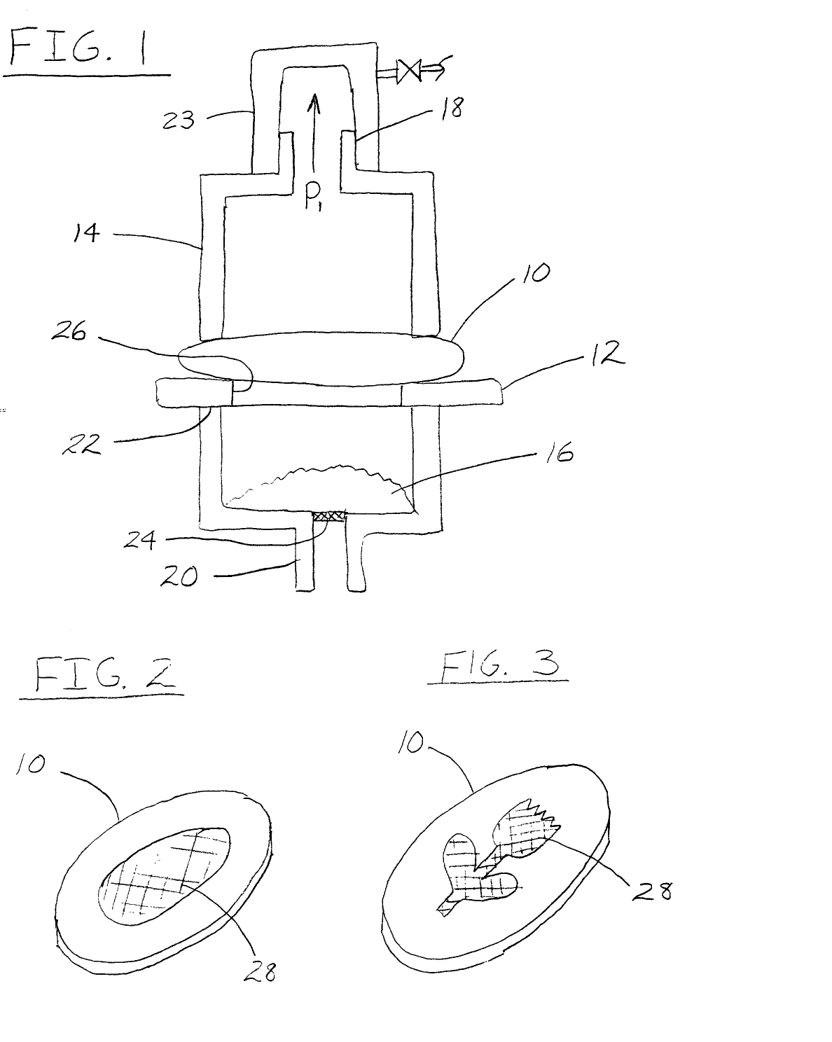

Referring to FIGS. 1-3, a pre-powdered cosmetic pad and the method for manufacturing same according to the present invention are shown.

A cosmetic pad 10 and a stencil 12 are inserted into a vacuum chamber 14 having an amount of cosmetic powder 16 deposited therein. The pad itself may be of any conventional design comprising a single layer or multiple layers assembled together. Suitable materials for the pad include foams made from such materials as polyester, polyethylene, polystyrene, polyurethane, polyvinylchloride, etc., flocked foams, woven or knitted velours, pile materials or sponges, provided that the material allows a quantity of powder to be forced therethrough. The material chosen should possess open cells or pores or loose weaves whose size depends on the grain size of the powder to embedded. The thickness of the pad of the present invention is generally between 1/16″ to ⅜″ and preferably between 3/32″ to ¼″ to allow sufficient penetration of the powder through the thickness of the pad.

As used herein, powder means a loose powder disbursed in a binder or compacted. The powders, which may be employed in the present invention, can be cosmetic, dermatological or pharmaceutical products for topical application.

The vacuum chamber 14 is generally an airtight container into which a desired amount of cosmetic powder 16 is deposited and a cosmetic pad 10 and a stencil 12 are inserted. The vacuum chamber 14 shown in FIG. 1 is a generally cylindrical container including an outlet 18, an inlet 20 and a cosmetic pad receiving space 22. Preferably, a vacuum source 23 is connected to the inlet 18 for providing a negative pressure to the chamber. In this case, the inlet 20 acts as a vent for allowing a flow of air through the chamber 14. This arrangement can be reversed, however, and a positive pressure source can be connected to the inlet 20, wherein the outlet 18 will act as a vent to allow a flow of air through the chamber 14.

The cosmetic pad receiving space 22 is essentially a slot extending transversely through opposite sides of the chamber wall at a location between the inlet 20 and the outlet 18. The cosmetic pad receiving space 22 shown in FIG. 1 is sized to receive both the pad 10 and the stencil 12 and is constructed such that the stencil 12 and cosmetic pad 10 are held at their outer peripheries by the wall of the vacuum chamber 14. However, a second slot can be provided in the chamber for insertion of the stencil 12 at a different location. In any event, it is important that the stencil 12 be inserted into the chamber 14 between the pad 10 and the cosmetic powder 16. Preferably, a filter 24 or fine mesh screen is connected to the vent 22 to prevent the escape of any powder 16.

The stencil 12 is made from a generally rigid material and includes a cutout portion 26, which may be formed in any desired shape or pattern. The stencil 12 is inserted into the vacuum chamber 14 between the cosmetic pad 10 and the cosmetic powder 16 contained within the chamber. However, if a predetermined pattern of embedded powder is not desired, it is possible to embed the cosmetic powder into the pad 10 without the use of the stencil 12 using the chamber 14 of the present invention.

Once the powder 16 has been deposited within the vacuum chamber 14 and cosmetic pad 10 and the stencil 12 have been secured in place, a vacuum source is connected to the outlet 18 and a vacuum (negative) pressure PI is applied to the surface of the pad opposite the stencil. The preferable vacuum pressure PI to be applied is varied and depends on the density of the powder and the porosity of the pad. The vacuum pressure PI causes a flow of air through the vacuum chamber 14 into which the cosmetic powder 16 becomes entrained. The flow of air with the entrained powder is drawn through the cut out portion 26 of the stencil 12 and through the cosmetic pad 10. As the powder 16 flows through the pad 10 it becomes embedded within the cells or pores of the pad. The desired amount of powder 16 to be embedded within the pad 10 depends upon the size of the cut-out portion 26 of the stencil 12, the density/porosity of the pad and the vacuum pressure and duration of vacuum applied.

Terminating the vacuum pressure P1 and removing the cosmetic pad 10 from the vacuum chamber 14 completes the process. FIGS. 2 and 3 illustrate cosmetic pads 10 formed in accordance with the present invention with different patterns of powder embedded in the surface of the pad. As shown in FIG. 3, any desired pattern including ornamental shapes, monograms or corporate logos may be embedded in the pad. Additionally, because the powder pattern is visible on the surface of the pad, the user is provided with convenient visual identification of the color of the powder product The pad having the powder pre-applied renders it unnecessary for the user to additionally carry a separate container for powder and enables powder to be applied more conveniently and expeditiously. To prevent the undesired emission of powder from the pad while being carried, an impermeable envelope or container may also be provided within which the cosmetic pad constructed in accordance with the invention may be placed. The pad may be designed for single use, wherein it is subsequently discarded, or it may be designed for multiple uses.

As a result of the present invention, a unique pre-powdered cosmetic pad having a desired powder pattern embedded on the surface of the pad and a simple cost efficient method of manufacturing the same is provided.

Although the illustrative embodiments of the present invention have been described herein with reference to the accompanying drawings, it is to be understood that the invention is not limited to those precise embodiments and that various other changes and modifications may be effected therein by one skilled in the art without departing from the scope or spirit of the invention.

What is claimed is:

1. A method of embedding an amount of cosmetic powder into a cosmetic pad comprising the steps of:
inserting a cosmetic pad into a chamber having an amount of cosmetic powder deposited therein; and
applying a pressure within the chamber whereby an amount of cosmetic powder is drawn into the cosmetic pad and embedded therein.

2. The method as defined in claim 1, wherein the pressure applied within the chamber is a negative pressure.

3. The method as defined in claim 1, wherein the pressure applied within the chamber is a positive pressure.

4. The method as defined in claim 1, further comprising the step of inserting a stencil into the chamber between the cosmetic pad and the amount of cosmetic powder whereby, upon the application of pressure within the chamber, an amount of cosmetic powder is drawn through the stencil and embedded into the cosmetic pad.

5. The method as defined in claim 4, wherein the stencil includes a decorative cutout pattern for embedding the cosmetic powder into the cosmetic pad in a decorative design.

6. An apparatus for embedding an amount of powder into a cosmetic pad comprising:
a chamber including an inlet, an outlet and a cosmetic pad receiving space disposed between said inlet and said outlet, said cosmetic pad receiving space being sized to receive at least a cosmetic pad inserted between said inlet and said outlet;
an amount of cosmetic powder deposited within said chamber between said inlet and said cosmetic pad receiving space; and
a pressure source connected to one or both of said inlet and said outlet for applying a pressure within said chamber to draw an amount of said cosmetic powder into a cosmetic pad inserted in said cosmetic pad receiving space.

7. The apparatus as defined in claim 6, further comprising a stencil disposed between said cosmetic pad receiving space and said amount of cosmetic powder, said amount of said cosmetic powder being drawn through said stencil.

8. The method as defined in claim 7, wherein said stencil includes a decorative cutout pattern for embedding the cosmetic powder into the cosmetic pad in a decorative design.

9. The method as defined in claim 6, wherein the pressure source applies a negative pressure to said outlet of said chamber.

10. The method as defined in claim 6, wherein the pressure source applies a positive pressure to said inlet of said chamber.

11. The apparatus as defined in claim 6, further comprising a screen disposed over at least one of said inlet and said outlet for preventing said cosmetic powder from escaping said chamber.

12. A pre-powdered cosmetic pad comprising:
a thin open-celled foam pad, said pad having a top face and a bottom face; and
an amount of cosmetic powder embedded into said top face of said pad, said cosmetic powder being visible on said top face of said pad.

13. The pad as defined in claim 12, wherein the cosmetic powder is embedded into said top face of said pad in a decorative pattern.

14. The pad as defined in claim 12, further including an impermeable envelope for containing the pad prior to use.
